(19) United States
(12) Patent Application Publication
(10) Pub. No.: US 2016/0324877 A1
(43) Pub. Date: Nov. 10, 2016

(54) COMBINATION OF VITAMIN D AND 25-HYDROXYVITAMIN D 3

(71) Applicant: DSM IP ASSETS B.V., Heerlen (NL)

(72) Inventors: Neil Robert BUCK, Leymen (FR); Wouter CLAERHOUT, Binningen (CH); Bruno H. LEUENBERGER, Rheinfelden (CH); Elisabeth STOECKLIN, Arlesheim (CH); Kai URBAN, Bad Sackingen (DE); Swen WOLFRAM, Waldshut-Tiengen (DE)

(21) Appl. No.: 15/214,666

(22) Filed: Jul. 20, 2016

Related U.S. Application Data
(62) Division of application No. 12/867,372, filed on Nov. 9, 2010, now abandoned.
(60) Provisional application No. 61/028,510, filed on Feb. 13, 2008.
(60) Provisional application No. 61/031,671, filed on Feb. 26, 2008.
(60) Provisional application No. 61/036,928, filed on Mar. 15, 2008.

(51) Int. Cl. A61K 31/593; A23L 33/155; A61K 9/16

(52) U.S. Cl. CPC A61K 31/593; A61K 9/16; A23L 33/155; A23V 2002/00

(57) ABSTRACT

We disclose compositions comprising Vitamin D (cholecalciferol and /or ergocalciferol) and 25-OH D3 (calcifediol), and use of those compositions to affect at least concentration, bioavailability, metabolism, or efficacy of vitamin D in a human. Forms and dosages of the composition, as well as processes for manufacturing a spray-dried formulation, are also disclosed.

### FIELD OF THE INVENTION

The present invention relates to a composition comprising Vitamin D (cholecalciferol/and/or ergocalciferol) and 25-hydroxyVitamin D3 (calcifediol), and use of that composition to affect at least concentration, bioavailability, metabolism, or efficacy of vitamin D.

### BACKGROUND OF THE INVENTION

Vitamin D (e.g., ergocalciferol and cholecalciferol) is a group of fat-soluble compounds defined by their biological activity. A deficiency of vitamin D causes rickets in children and osteomalacia in adults. But toxicity can occur after chronic intake of more than 100 times the recommended daily allowance (i.e., 5-15 μg or 200-600 IU vitamin D) for several months. For vitamin D, “The threshold for toxicity is 500 to 600 mcg/kg body weight per day. In general, adults should not consume more than three times the RDA for extended period of time” (Garrison & Somer, The Nutrition Desk Reference, Third Ed., McGraw-Hill, pg. 82, 1997). Hypercalcemia may occur at a blood concentration of 25-hydroxyvitamin D greater than 375 nmol/L. More recently, a safe upper level of Vitamin D was identified to be at least 250 μg/day (10′000 IU) (Hathcock et al. Am.J Clin. Nutr. 85:6-18, 2007). Ingestion of such as a dietary supplement has been shown to result in a blood concentration of about 200 nmol/L 25-hydroxyvitamin D.

Vitamin D is a prohormone which has to be hydroxylated in the liver to produce 25-hydroxyvitamin D (calcifediol; 25-OH vitamin D; 25-OH D), which then undergoes another hydroxylation in the kidney and other tissues to produce 1,25-dihydroxyvitamin D, the active hormone form of vitamin D. 1,25-dihydroxyvitamin D is released into the blood, binds to vitamin D binding protein (DBP), and is transported to target tissues. Binding between 1,25-dihydroxyvitamin D and vitamin D receptor allows the complex to act as a transcription factor in the cell's nucleus.

Vitamin D deficiency may promote resorption of bone. It may also modulate function of the cardiovascular, immune, and muscular systems. Epidemiological studies find associations between vitamin D intake and its effect on blood pressure or glucose metabolism. The activity of vitamin D is under negative feedback control by parathyroid hormone.

Both Vitamin D and 25-OH D3 have been administered as pharmaceuticals in the past. Vitamin D, is of course widely available; 25-OH D3 was previously sold in the USA by Organon USA under the name “CALDEROL”, but is currently on the FDA's list of discontinued drugs. It was a gelatine capsule containing corn oil and 25-OH D3.

A liquid form of 25-OH D3 is currently sold in Spain by FAES Farma under the name “HIDROFEROL” in an oil solution.

The combination of vitamin D and 25-OH D3 has been used in animal feed. 25-OH D3 for use in feed is commercially available from DSM under the name “ROVIMIX HY-D”.

Tritsch et al. (US 2003/0170324) disclose a feed premix composition of at least 25-OH D3 in an amount between 5% and 50% (wt/wt) dissolved in oil and an antioxidant, an agent encapsulating droplets of 25-OH D3 and oil, and a nutritional additive (e.g., Vitamin D3). The premix may be added to poultry, swine, canine, or feline food. This composition stabilizes 25-OH D3 against oxidation.

Simoes-Nunes et al. (US 2005/0064018) discloses adding a combination of 25-OH Vitamin D3 and Vitamin D3 to animal feed. In particular, about 10 μg/kg to about 100 μg/kg of 25-OH Vitamin D3 and about 200 IU/kg to about 4,000 IU/kg of Vitamin D3 are added to swine feed. This addition improves the pig's bone strength.

Stark et al. (U.S. Pat. No. 5,695,794) disclose adding a combination of 25-OH Vitamin D3 and Vitamin D3 to poultry feed to ameliorate the effects of tibial dyschondroplasia.

Borenstein et al U.S. Pat. No. 5,043,170 discloses the combination of Vitamin D3 and either 1-alpha-hydroxycholecalciferol or 1 alpha, 25-dihydroxycholecalciferol to improve egg strength and leg strength in laying hens and older hens.

Chung et al, WO 2007/059960 discloses that sows fed a diet containing both Vitamin D3 and 25-hydroxVitamin D3 had improved general health status, body frame, litter size and health, and other production parameters. Also a 25-OH D3 human food supplement is disclosed, but its dosage range, 5-15 micrograms per kg body weight, which equals to an extremely high daily dosage of 300-900 micrograms per human is very high.

The aforementioned documents did not teach or suggest use of a combination of Vitamin D and 25-OH D3 as a pharmaceutical, food, or nutraceutical for humans or its effects on human health. Forms and dosages of this composition provide desirable effects on the use of vitamin D by humans (e.g., pharmacokinetics). The kinetics of dosing with both Vitamin D and 25-OH D3 in humans has not been previously studied to our knowledge. Other advantages and improvements are described below or would be apparent from the disclosure herein.

### DETAILED DESCRIPTION OF THE INVENTION

It has been surprisingly found that the increase of plasma 25-OH D increases synergistically when a person is administered a combination of Vitamin D and 25-OH D3. This effect is observed rapidly, and is most pronounced after about the first 6 hours. Further, the increase in plasma levels is sustained over approximately 206 hours. Thus, this invention comprises a combination of Vitamin D (cholecalciferol and/or ergocalciferol) and 25-OH D3 (calcifediol) for use as a pharmaceutical in a human. One possible use of such a composition is as an anti-osteoporosis agent, although the combination of this invention is suitable for any indication where a Vitamin Dor 25-OH D deficiency is implicated.

Further, it has been found, in accordance with this invention that the combination of 25-OH D3 and Vitamin D synergistically regulates (either up-regulates or down-regulates) a synergistic number of Vitamin D responsive genes, including a high number of genes which are not responsive to the presence of either Vitamin D nor 25-OH D3 alone. This is a surprising result, as it is not explained by the current model of Vitamin D metabolism, which postulates that virtually all Vitamin D is first metabolized into 25-OH D.

The combination, in accordance with this invention, provides two significant advantages:

1) It results in a rapid and synergistic plasma response of 25-OH D;

2) It leads to an unexpectedly pronounced and long plateau of plasma 25-OH D levels. These are especially important goals of treatment of Vitamin D deficiency: a rapid correction of suboptimal Vitamin D status and a long and stable plasma concentration to ensure sufficient supply of all Vitamin D dependent tissues.

Another aspect of this invention is a food, functional food, food supplement or nutraceutical suitable for human consumption containing 25-OH D3, and preferably a combination of Vitamin D and 25-OH D3.

### BRIEF DESCRIPTION OF THE FIGURES

FIG. 1 shows Venn Diagrams of differentially expressed probe sets for murine genes for the hindlimb unloaded (“HU group”) and the treatment groups (Vitamin D3, 25-OH D3 or the combination).

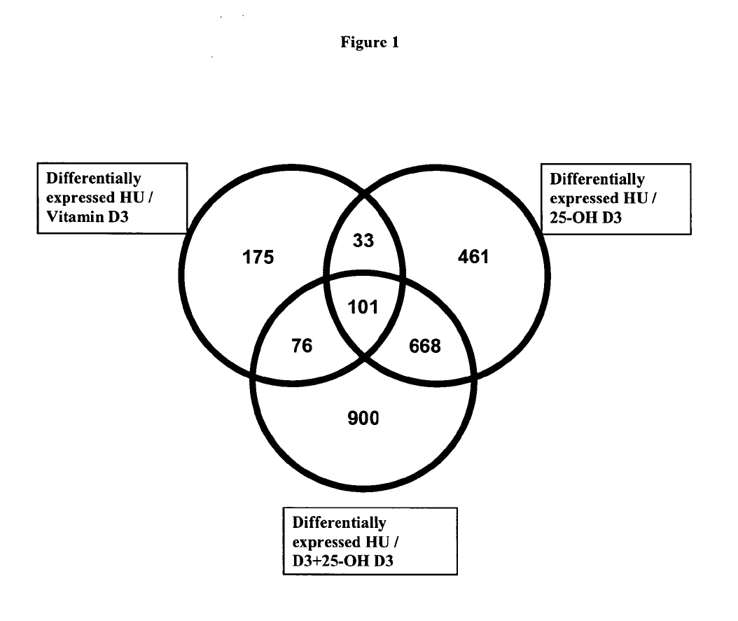

FIG. 2 shows Venn Diagrams of differentially expressed probe sets for genes between 25-OH D3 treatment group and the treatment group with the combination of 25-OH D3+Vitamin D3.

FIG. 3 shows Venn Diagrams of differentially expressed probe sets for genes between Vitamin D3 treatment group and the treatment group with the combination of 25-OH D3+Vitamin D3.

FIG. 4 is an enrichment analysis (performed with GeneGo MetaCore) of the 1745 probe sets for genes differentially expressed between the HU group and the group receiving a combination treatment of 25-OH D3 and Vitamin D3.

FIG. 5 shows Venn Diagrams of differentially expressed probe sets in the 25-OH D3+Vitamin D3 treatment group and probe sets for selected skeletal muscle genes.

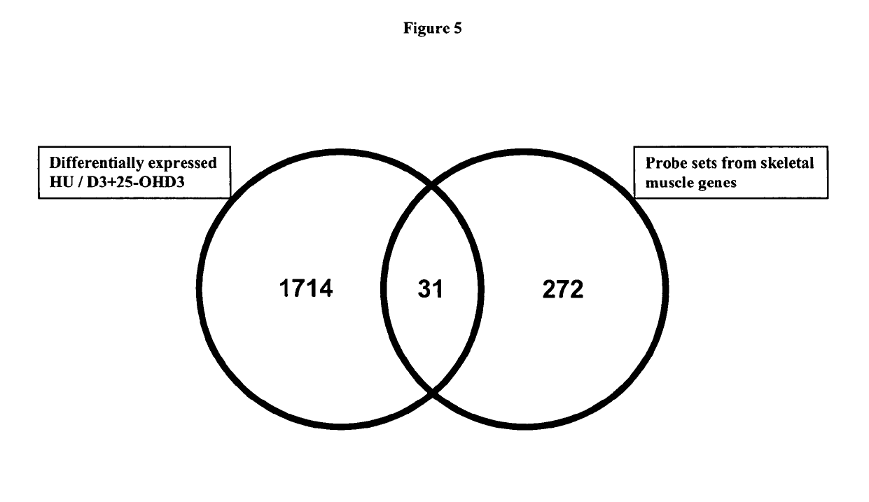

FIG. 6 shows Venn Diagrams of differentially expressed probe sets in the 25-Hydroxyvitamin D3 treatment group and probe sets for selected skeletal muscle genes.

FIG. 7 showsVenn Diagrams of the differentially expressed probe sets in the Vitamin D3 treatment group and probe sets for selected skeletal muscle genes.

As used throughout the specification and claims, the term “Vitamin D” means either Vitamin D3 (cholecalciferol) and/or Vitamin D2 (ergocaciferol). Humans are unable to make Vitamin D2 (ergocalciferol), but are able to use it as a source of Vitamin D. Vitamin D2 can be synthesized by various plants and is often used in Vitamin D in supplements as an equivalent to Vitamin D3.

“Vitamin D metabolite” means any metabolite of Vitamin D other than 25-hydroxy vitamin D3.

“25-OH D3” refers specifically to 25-hydroxyvitamin D3

“25-OH D” refers to the 25-hydroxylated metabolite of either Vitamin D2 or Vitamin D3 which is the major circulating form found in plasma.

In one embodiment, a pharmaceutical composition suitable for human use is provided which comprises Vitamin D, 25-OH D3, and a pharmaceutically acceptable antioxidant, and at least one pharmaceutically acceptable carrier in tablet, capsule, or injectable form.

This invention is also drawn to a human pharmaceutical composition wherein the active ingredients consist essentially of a combination of Vitamin D and 25-OH D3; and more preferably, the active ingredients consist essentially of a combination of Vitamin D3 and 25-OH D3.

In another embodiment, a kit is provided which is comprised of multiple, separate dosages of Vitamin D or Vitamin D3 along with a dosage of 25-OH D3. They may be enclosed in a container: e.g., bottle, blister pack, or vial rack. Further, instructions for administering the composition as a dosage to a human are provided within the kit.

In another embodiment, a composition suitable for human use is provided which is comprised of at least a spray-dried formulation of 25-OH D3 and a pharmaceutically acceptable antioxidant. The composition may be further comprised of spray-dried formulation of Vitamin D or Vitamin D3. Alternatively, the composition may be a mixture of Vitamin D or Vitamin D3 and 25-OH D3 and a pharmaceutically acceptable antioxidant from which a spray-dried formulation is prepared. Or a kit is provided which is comprised separately of a first composition of at least a spray-dried formulation of Vitamin D or Vitamin D3 and a pharmaceutically acceptable antioxidant and a second composition of at least a spray-dried formulation of 25-OH D3.

In yet another aspect, a process for manufacturing a spray-dried formulation is provided. At least Vitamin D, 25-OH D3, or both are dissolved in a suitable oil such as medium chain triglycerides, coconut oil, or palm oil to provide a nonaqueous phase. The nonaqueous phase and an aqueous phase are emulsified. The emulsion is sprayed and dried to a powder. Alternatively, Vitamin D or Vitamin D3 and 25-OH D3 are separately spray dried to a powder, then mixed with each other.

In another aspect, a method of administering Vitamin D and 25-OH D3 to a human is provided. Thereby, concentration of 25-OH D may be increased or maintained in blood, plasma, or serum; a steady-state concentration of 25-OH D may be achieved in blood, plasma, or serum; the predictability of the plasma or serum concentration of 25-OH D may be increased.

The peak concentration of 25-OH D achieved by such administration may be from 30 nmol/L to 375 nmol/L, preferably from about 120 nmol/L to about 300 nmol/L. The steady-state concentration of 25-OH D achieved by such administration is preferably from above 60 nmol/L.

In another embodiment, the 25-OH D3, alone or in combination with Vitamin D is the active ingredient in a food, functional food, food supplement or nutraceutical suitable for human consumption. The dosages of the 25-OH and/or D3 may be the same as those present in the pharmaceutical product, but preferably will tend towards the lower ranges. The food supplements and nutraceuticals may be in the form of tablets, capsules or other convenient dosage forms. The food may be a beverage or food, and if desired, may also contain other nutritionally effective compounds such as other vitamins, minerals, and the like.

Vitamin D deficiency is an especially prevalent condition in the elderly population and those who suffer chronic immobility regardless of age. This may be due to the general lack of exposure to sunlight, a lessened ability of the body to manufacture vitamin D or metabolize it efficiently, or a number of other causes. Thus one aspect of this invention is the use of the combination of Vitamin D and 25-OH D3 in an elderly population. As used throughout, the term “elderly” is meant to encompass those individuals who are over 65 years of age, preferably over 70, and even over 80.

In another embodiment, this combination of 25-OH D3 and Vitamin D is suitable for people who are at risk of developing conditions characterized by Vitamin D deficiency or insufficiency. This would include especially adults, including post-menopausal women (i.e.

about age 45 and older) and men who are about age 45 and older. It is especially suitable for individuals who do not receive a great deal of natural sunlight exposure, such as for people who traditionally wear long clothing, do not go out of doors regularly, or who use sunscreens when they are exposed to sunlight, or live in geographical areas significantly north or south of the equator, where sunlight is less intense.

The combination of vitamin D and 25-OH D3 may be administered once per day, once per week, or once per month. It has been surprisingly found, in accordance with this invention, that by delivering a combination of the vitamin D and 25-OH D3, plasma levels of 25-OH D increase synergistically. This effect is observed rapidly, and is most pronounced after about the first 6 hours. Further, the increase in plasma levels is sustained (albeit at a lower, but still clinically effective level), for at least approximately 206 hours. Thus this invention offers two distinct advantages over prior treatments of the single Vitamin D forms: The rapid effect provides acute bioavailability, while the sustained elevated plasma levels ensures extended bioavailability.

In still another aspect, a method of using a spray-dried formulation of at least 25-OH D3 is provided by administering it to a human.

Another aspect of this invention is a method of providing vitamin D or its metabolites to a human with a malabsorption syndrome (e.g., affected by celiac disease, sprue, or short bowel syndrome) by administering the combination of Vitamin D and 25-OH D3.

Another aspect of this invention is a method of providing vitamin D metabolites to a human with impared liver function, wherein the human cannot process Vitamin D into 25-hydroxyvitamin D efficiently by providing the human with a combination of Vitamin D and 25-hydroxyvitamin D3.

Vitamin D and 25-OH D3 may be obtained from any source, and a composition thereof may be prepared using convenient technology. In general, crystals of Vitamin D, 25-OH D3, or both (separately or together) are dissolved in an oil with heating and agitation. Preferably, the oil is transferred into a vessel and heated. Thereafter, Vitamin D, 25-OH D3, or both are added to the vessel, while maintaining the temperature of the oil or increasing it over time. The composition is agitated to dissolve the crystals of Vitamin D, 25-OH D3, or both. Prior to addition to the oil, the crystals may be reduced in size by milling and/or sieving, to enhance dissolving. The composition may be agitated by stirring, vessel rotation, mixing, homogenization, recirculation, or ultrasonication. Preferably, the oil may be heated in the vessel to a temperature from about 80° C. to about 85° C.; sized crystals are introduced into the vessel, and the contents are stirred to dissolve the crystals into the oil.

The “oil” may be any suitable edible oil, lipid, or fat: e.g., babassu oil, coconut oil, cohune oil, murumyru tallow, palm kernel oil, or tucum oil. The oil may be natural, synthetic, semisynthetic, or any combination thereof. Natural oil may be derived from any source (e.g., animal, plant, fungal, marine); synthetic or semisynthetic oil may be produced by convenient technology. Preferably, the oil is a mixture of plant medium chain triglycerides, mainly caprylic and capric acids, or palm or coconut oil or mixtures thereof. The composition may optionally contain one or more other suitable ingredients such as, for example, and a pharmaceutically acceptable antioxidant, preservatives, dissolution agents, surfactants, pH adjusting agents or buffers, humectants, and any combination thereof. The foregoing are examples of pharmaceutically acceptable carriers.

Suitable antioxidants include tocopherol, mixed tocopherols, tocopherols from natural or synthetic sources, butylated hydroxy toluene (BHT), butylated hydroxy anisole (BHA), natural antioxidants like rosemary extract, propyl galate, and any others used in the manufacture of pharmaceuticals for humans. Preferably, the antioxidant is tocopherol. Suitable preservatives include methyl paraben, propyl paraben, potassium sorbate, sodium benzoate, benzoic acid, and any combination thereof. Suitable dissolution agents include inorganic or organic solvents: e.g., alcohols, chlorinated hydrocarbons, and any combination thereof. Suitable surfactants may be anionic, cationic, or nonionic: e.g., ascorbyl palmitate, polysorbates, polyethylene glycols, and any combination thereof. Suitable pH adjusting agents or buffers include citric acid-sodium citrate, phosphoric acid-sodium phosphate, acetic acid-sodium acetate, and any combination thereof. Suitable humectants include glycerol, sorbitol, polyethylene glycol, propylene glycol, and any combination thereof.

Once formed, the oil composition may be incorporated in various other useful compositions, some of which are discussed below. For example, emulsions may be formed, which may be optionally encapsulated or spray dried. A variety of emulsions may be prepared by combining the nonaqueous compositions described above with an aqueous composition. The emulsion may be of any type. Suitable emulsions include oil-in-water emulsions, water-in-oil emulsions, anhydrous emulsions, solid emulsions, and micro emulsions.

The emulsions may be prepared by any convenient technology. The emulsion contains an aqueous composition and a nonaqueous (e.g., oil) composition, wherein the latter comprises Vitamin D, 25-OH D3, or both (separately or together) dissolved in an oil in an amount of between about 3% and about 50% by weight based on the total weight of the oil composition. As used herein, “aqueous composition” and “aqueous phase” are used interchangeably. Generally, the emulsion may contain from about 20% to about 95% of an aqueous composition, and from about 5% to about 80% of a nonaqueous composition. Preferably, however, the emulsion contains from about 85% to about 95% (vol/vol) of an aqueous composition, and from about 5% to about 15% (vol/vol) of a nonaqueous composition. Conveniently, the nonaqueous composition may be dispersed as droplets in the aqueous composition. For example, the droplets may have a mean diameter of less than about 500 nm in the aqueous composition. Conveniently, the droplets have a mean diameter of between about 150 nm and about 300 nm.

In a particularly advantageous embodiment, the emulsion contains an encapsulating agent, which facilitates encapsulating the oil composition upon further processing of the emulsion (e.g., by spray drying). The encapsulating agent may be any edible substance capable of encapsulating the oil composition. Preferably, the encapsulation agent is predominantly a colloidal material. Such materials include starches, proteins from animal sources (including gelatins), proteins from plant sources, casein, pectin, alginate, agar, maltodextrins, lignin sulfonates, cellulose derivatives, sugars, saccharides, sorbitols, gums, and any combination thereof.

Suitable starches include: plant starches (e.g., CAPSUL® or HI-CAP® from National Starch & Chemical Corp., New York, N.Y.), other modified food starches, and any combination thereof. Preferably, the starch is CAPSUL® modified plant starch. Suitable proteins from animal sources include: gelatins (e.g., bovine gelatins, porcine gelatins (Type A or B) with different Bloom numbers, fish gelatins), skim milk protein, caseinate, and any combination thereof. Preferably, the animal protein is a gelatin. Suitable proteins from plant sources include: potato protein (e.g., ALBUREX® from Roquette Preres Societe Anonyme, Lestrem, France), pea protein, soy protein, and any combination thereof. Preferably, the plant protein is ALBUREX® potato protein. Suitable maltodextrins with a different dextrose equivalent include: maltodextrin 5, maltodextrin 10, maltodextrin 15, maltodextrin 20, maltodextrin 25, and any combination thereof. Preferably, the maltodextrin is maltodextrin 15. Suitable cellulose derivatives include: ethyl cellulose, methylethyl cellulose, hydroxypropyl cellulose, hydroxypropylmethyl cellulose, carboxymethylcellulose, and any combination thereof. Suitable saccharides include lactose, sucrose, or any combination thereof. Preferably, the saccharide is sucrose. Suitable gums include: acacia, locust bean, carragean, and any combination thereof. Preferably, the gum is gum acacia.

When the emulsion contains an encapsulating agent, the encapsulating agent may be dispersed in water by any convenient technology to form an aqueous phase. The aqueous phase may be a solution or a mixture depending on the properties of the components selected. The selected components may be dispersed by any convenient technology including: homogenizing, mixing, emulsifying, recirculating, static mixing, ultrasonication, stirring, heating, or any combination thereof. The viscosity of the resulting aqueous phase may then be adjusted, as desired, by the addition of water. The aqueous composition of the emulsion may optionally contain any other suitable material including but not limited to, those discussed above in reference to the nonaqueous composition. Preferably, the aqueous composition may include, an encapsulating agent, a film-forming agent, a plasticizer, a preservative, an antioxidant, or any combination thereof. Suitable preservatives include methyl paraben, propyl paraben, sorbic acid, potassium sorbate, sodium benzoate, and any combination thereof. Suitable antioxidants include sodium ascorbate, ascorbic acid, citric acid, and any combination thereof.

Preferably, the aqueous phase contains a modified food starch, such as octenyl succinyl starch (CAPSUL®), maltodextrin, and sodium ascorbate. Another preferred aqueous phase contains potato protein (ALBUREX®), maltodextrin 20, and sodium ascorbate. The selected components may be dissolved in water by any convenient technology, preferably stirring. The mixture is preferably homogenized until it is uniform and lump free. Preferably, the homogenization is carried out at a temperature between about 50° C. and about 80° C. The final viscosity of the resulting aqueous phase may then be adjusted to the desired viscosity, preferably about 250 mPa·s to about 450 mPa·s, more preferably about 300 mPa·s to about 400 mPa·s, even more preferably about 385 mPa·s, for beadlet production, or 60 mPa·s to 300 mPa·s, for spray drying formulations.

The emulsion may be formed by emulsifying the nonaqueous composition and the aqueous phase by any means, including homogenization, rotor-stator shear, high pressure shear and cavitation, high speed “cowles” or shear agitation, and any combination thereof. The volume and viscosity of the emulsion may preferably be adjusted by the addition of water after emulsification. Preferably, the nonaqueous and aqueous compositions are emulsified by homogenization. Preferably, the emulsion should not contain any mineral, transition metal, or peroxide.

As noted above, the emulsion may be incorporated or employed in producing other useful compositions, especially encapsulated oils, e.g., spray-dried powders. Generally, the encapsulated oil comprises an oil composition and an encapsulation agent encapsulating the oil composition, wherein the oil composition contains Vitamin D, 25-OH D3, or both dissolved in the oil in an amount between about 5% and about 50% by weight based on the total weight of the oil composition. The encapsulated oil may be produced by any convenient technology: e.g., drying an emulsion described above by any conventional technology, including spray drying, freeze drying, fluid bed drying, tray drying, adsorbtion, and any combination thereof. Preferably, the encapsulated oil is produced by spray drying an emulsion having an aqueous phase above containing an encapsulation agent; spray drying parameters are dictated by the physical characteristics desired in the final encapsulated oil. Such physical parameters include particle size, powder shape and flow, and water content. Preferably, the oil is in an amount less than about 30%, less than about 20%, less than about 10%, or less than about 5% by weight based on the total weight of the encapsulated oil. The encapsulated oil should have good flowability and the Vitamin D and/or 25-OH D3 should be distributed homogeneously throughout the composition. Conveniently, the encapsulated oil is a powder. Any other suitable additive may be added to the encapsulated oil. One such additive may be a flow agent such as silicon dioxide, to increase the flowability of the encapsulated oil.

The composition may be provided in the form of a tablet, capsule (e.g., hard or soft), or injection (e.g., oil or emulsion). They may be packaged in a single daily dosage.

### Dosages

Daily. A composition according to this invention where the two active ingredients are to be administered separately, contains Vitamin D or 25-OH D3 in an amount from about 1 μg to about 50 μg, preferably about 5 μg and 25 μg. Alternatively, a single daily dosage having both Vitamin D and 25-OH D3 contains each active ingredient in an amount from about 1 μg to about 50 μg, preferably about 5 μg and 25 μg.

The dosage ratio of Vitamin D to 25-OH D3 may be from about 50:1 to about 1:50, more preferably from about 25:1 to about 1:25, and even more preferably from about 6:1 to about 1:6.

Multiple, separate dosages may be packaged in a single kit (or container). For example, the kit may be comprised of thirty separate daily dosages of both actives separately (i.e. 60 separate dosages), or combined (i.e. 30 dosages containing both active ingredients). Instructions for administering the dosages to a human may be included in the kit.

Weekly. A single weekly dosage contains Vitamin D or 25-OH D3 in an amount from about 7 μg to about 350 μg, and preferably from about 35 to 175 pg. Alternatively, a single weekly dosage may contain both Vitamin D and 25-OH D3 each in an amount from about 7 μg to about 350 μg, and preferably from about 35 to 175 μg . The dosage ratio of Vitamin D to 25-OH D3 may be from about 50:1 to about 1:50, more preferably from about 25:1 to about 1:25, and even more preferably from about 6:1 to about 1:6.

Monthly. A single monthly dosage contains Vitamin D or 25-OH D3 in an amount from 30 μg to about 1500 μg, preferably about 75 μg to about 500 μg. Alternatively, a single monthly dosage may contain both Vitamin D and 25-OH D3 each in an amount from 30 μg to about 1500 μg, preferably about 75 μg to about 500 μg. A kit may be comprised of one, two, three, four, five, six, seven, eight, nine, ten, eleven, or twelve weekly or monthly dosages.

Dosage ratios of Vitamin D to 25-OH D3 should range between 50:1 to about 1:50, more preferably from about 25:1 to about 1:25, and even more preferably from about 6:1 to about 1:6.

It has been found that a dosage ratio of approximately 1:6 25-OH D3 to D3 is particuarly beneficial in increasing plasma 25-OH D levels quickly (i.e within a few hours) and maintaing elevated plateau levels.

Thus another aspect of this invention is a method of maintaining a prolonged plasma level of at least 60 nmol/L of 25-OH D above the baseline level by administering a combination of Vitamin D and 25-OH D3. Preferably the Vitamin D is Vitamin D3.

As used throughout this application and claims, “prolonged” means the time period which starts at four hours after ingestion of Vitamin D and 25-OH D3, and lasts until at least 12 hours.

Another aspect of this invention is a method of maintaining an increased plasma levels of 25-OH D by at least 30 nmol/L above the baseline level for at least one week by administering a combination of 25-OH D3 and Vitamin D. Preferably the Vitamin D is Vitamin D3.

Another aspect of this invention is a method of increasing plasma levels of 25-OH D by at least 30 nmol/L above baseline levels for at least one week by administering a combination of 25-OH D3 and Vitamin D. Preferably the Vitamin D is Vitamin D3.

Another aspect of this intervention is a method of increasing plasma levels of 25-OH D by at least 30 nmoUL above baseline levels acutely, i.e. within 2 hours after administering a combination 25-OH D3 and Vitamin D and maintaining plasma levels of at least 30 nmol/L above baseline for at least one week. Preferably the Vitamin D is Vitamin D3.

As can be appreciated, one of the advantages of the administration of both 25-OH D3 and Vitamin D, preferably Vitamin D3, is that the circulating amount of 25-OH D rapidly increases, and that this increase is sustained. If one administers lower dosages, such as those recommended for a daily regime, the absolute increase in plasma, will of course be lower than than demonstrated in the higher dose in the Example, i.e the increase will be less than 30 nmol/l. However, the overall response pattern is the same, i.e. there is a rapid increase and a long plateau wherein an increase over baseline is maintained. Even though the magnitude of the response is lower, there is still the advantage that plasma levels are stable throughout the day. Thus, all tissues are optimally supplied with 25-OH D.

Another advantage of this invention is that by administration of both Vitamin D and 25-OH D3, preferably Vitamin D3, the levels of circulating 25-OH D can be more easily reach a predetermined level and that this predetermined level can be sustained for a predictable length of time. Thus, the number of individuals who do not respond to Vitamin D therapy can be minimized For example, individuals with impaired liver functions, or similar conditions can now have more normalized 25-OH D levels.

Gene Analysis

To demonstrate increased bioactivity of the combination, a gene chip analysis of muscle tissue exposed to Vitamin D, 25-OH D3 and the combination was performed. Details are given in Example 2, using a murine hind-leg suspension model. As can be seen, there is a dramatic increase in the number of genes activated or regulated (either up-regulated or down-regulated) when the combination of the two are delivered as compared to individual administration. As it is currently believed that the vast majority of Vitamin D is converted into 25-OH D upon ingestion and processing in the liver, this is a surprising result.

Thus, another aspect of this invention is a process of activating or regulating Vitamin D and 25-OH D responsive human genes comprising administering to a person a combination of Vitamin D and 25-OH D3.

The following non-limiting Examples are presented to better illustrate the invention.

### EXAMPLES

### Example 1

### Formulation & Clinical Trial

### Formulation

### Materials and Methods

Spray-dried formulation of 25-OH D3 was provided as a powder. In summary, 25-OH D3 and DL-a-tocopherol were dissolved in an oil of medium chain triglycerides, then emulsified into an aqueous solution of modified starch, sucrose, and sodium ascorbate. The emulsion was atomized in a spray dryer in the presence of silicon dioxide. The resulting powder was collected when water content (LOD) was less than 4% and sieved through 400 μm. It was packed and sealed in alu-bags, then stored in a dry area below 15° C. and used within 12 months of its manufacture.

Three separate lots were manufactured. In detail, a matrix was produced by mixing for 120 min in a FRYMIX processing unit with an anchor stirrer at 70° C. under vacuum and consisting of:

17.300 kg water (WBI)

13.460 kg modified food starch (CAPSUL HS)

3.270 kg sucrose

0.730 kg sodium ascorbate

An oil phase was prepared by mixing for 35 min in a double-walled vessel with propeller stirrer at 82° C. and consisting of:

0.550 kg BERGABEST MCT oil 60/40

0.049 kg calcifediol (HY-D USP)

0.183 kg DL-a-tocopherol

The oil phase was transferred to the matrix in the FRYMIX processing unit and was pre-emulsified with its internal colloid mill (60 min, 70° C.). The pre-emulsion was circulated through a high-pressure homogenizer (20 min). The emulsion with a viscosity of 60 mPa·s to 90 mPa·s at 70° C. was transferred over the high pressure pump to the spray nozzle. As fluidizing agent, silicon dioxide (SIPERNAT 320 DS) was fed into the tower, although it is envisioned that other silicon dioxide forms may also be suitable. The spraying and drying parameters are listed below.

| Parameter | Spraying | | Drying | |
|---|---|---|---|---|
| Inlet air position | top of tower | | top of tower | |
| Inlet air feed | 1500 | m³/h | 1400 | m³/h |
| Inlet air temperature | 170° | C. | heater switch off | |
| IFB inlet air feed | 500 | m³/h | 500 | m³/h |
| IFB inlet air temperature | 65° | C. | 50° | C. |

| exhaust air position | bottom of the tower | | bottom of the tower |
|---|---|---|---|
| fine powder recycling | to IFB | | to IFB |
| emulsion feed rate | 50 | kg/h | Emulsion feed stopped |
| SiO₂ feed position | top of tower | | SiO₂ feed stopped |
| SiO₂ acid feed rate | 100 | g/h | SiO₂ feed stopped |

For each of the three lots of 25-OH D3, an average of 8.4 kg of spray-dried powder with about 0.25% content of 25-OH D3 was obtained. The other components of the formulation are: 73.2% modified food starch, 17.6% sucrose, 4.0% sodium ascorbate, 3.0% medium chain triglycerides, 1.0% silicon dioxide, and 1.0% DL-a-tocopherol.

Spray-dried formulation of Vitamin D3 was provided as a powder. In summary, Vitamin D3 and DL-a-tocopherol were dissolved in an oil of medium chain triglycerides, then emulsified into an aqueous solution of modified starch, sucrose, and sodium ascorbate. The emulsion was atomized in a spray dryer in the presence of silicon dioxide. The resulting powder was collected when water content (LOD) was less than 4% and sieved to remove big lumps. It was stored in a dry area below 15° C. Stability was good, and shelf life can be extended over 12 months.

### Clinical Trial

### Subjects

Healthy, postmenopausal women (50 to 70 years of age) were recruited using informed consent and screened using the following criteria: serum 25-OH D between 20 nmol/L and 50 nmol/L, body mass index between 18 kg/m² and 27 kg/m², blood pressure less than 146/95 mm Hg, serum calcium less than 2.6 nmol/L, fasting glucose less than 100 mg/dl, no high-intensity exercise more than three times per week, no treatment for hypertension, no use of high-dose vitamin D or calcium supplement or drug affecting bone metabolism (e.g., biphosphonate, calcitonin, estrogen receptor modulator, hormone replacement therapy, parathyroid hormone), and not visiting a “sunny” location during the study.

Subjects were randomly assigned to one of seven treatment groups (i.e., daily, weekly, bolus as single dose, and bolus as combination dose). Each group included five subjects. They are followed for four months in Zürich, Switzerland during the winter.

### Clinical Study

The objective was studying and comparing the pharmacokinetic characteristics of Vitamin D and 25-OH D3 administered to humans. Equal quantities of both substances were investigated. The regimen is based on 20 μg/day (or its equivalent on a weekly basis) of 25-OH D3. As the maximum pre-existing baseline concentration of 25-OH D will be 50 nmol/L, it is not anticipated that subjects will approach the range where disturbance in Ca²⁺ homeostasis has been observed. For comparative purposes, it was necessary to administer equimolar quantities of either Vitamin D or 25-OH D3. In respect to administration of Vitamin D, the dose is considered to be sufficient to overcome background variability and provide and efficacious dose to the participants.

Daily: 120 administrations

| | | | |
|---|---|---|---|
| 1. | 25-OH D3 | 20 | μg |
| 2. | Vitamin D3 | 20 | μg (800 IU) |

Weekly: 16 administrations

| | | | |
|---|---|---|---|
| 3. | 25-OH D3 | 140 | μg |
| 4. | Vitamin D3 | 140 | μg (5600 IU) |

Bolus: single administration

| | | | |
|---|---|---|---|
| 5. | 25-OH D3 | 140 | μg |
| 6. | Vitamin D3 | 140 | μg (5600 IU) |

Bolus: combo administration

| | | |
|---|---|---|
| 7. | D3 and 25(OH)D3 | 140 μg (5600 IU) + 140 μg |

Hard gel capsules, which are packaged in bottles, contain either 20 μg or 140 μg of either spray-dried Vitamin D or 25-OH D3 per capsule. Each dosage is consumed orally at breakfast. The duration of the study is four months for the “Daily” and “Weekly” groups. Subjects enrolled in the “Bolus” group consume orally a single dosage at the second study visit and are further followed-up for four months.

Plasma concentrations of 25-OH D (e.g., peak and steady state) were determined by obtaining samples from the subjects at various times after the dosage is ingested. For screening purposes and to establish baseline values, a blood sample is obtained prior to enrollment into the study and the clinical laboratory measures Vitamin D, 25-OH D3, calcium, creatinine, albumin, and fasting glucose in the serum. On Monday of Week 1 of the study, pharmacokinetics of serum Vitamin D, 25-OH D, and 1,25-dihydroxy Vitamin D; serum markers (i.e., Vitamin D, 25-OH D, calcium, creatinine, albumin, PTH, GOT, GPT, ALP, triglycerides, HDL, LDL, total cholesterol, bALP, and fasting glucose); and urine markers (i.e., calcium, creatinine, and DPD) are assessed over 24 hours. Daily samples for the remaining days of Week 1 and Monday of Week 2 are taken to assess serum Vitamin D and 25-OH D, serum markers (i.e., calcium, creatinine, albumin), and urine markers (i.e., calcium, creatinine). The assessments continue on Monday of Weeks 3, 5, 7, 9, 11, 13 and 15. On Monday of Week 16, samples are taken to assess pharmacokinetics of serum Vitamin D, 25-OH D, and 1,25-dihydroxy Vitamin D; serum markers (i.e., Vitamin D, 25-OH D, calcium, creatinine, albumin, PTH, GOT, GPT, ALP, triglycerides, HDL, LDL, total cholesterol, bALP, and fasting glucose); and urine markers (i.e., calcium, creatinine, and DPD).

Results

TABLE 1 shows the increase in plasma 25-OH D levels after a dosage of 140 μg 25-OH D3, a dosage of 140 μg Vitamin D or the combined dosage of 140 μg 25-OH D3+140 μg Vitamin D. Blood samples were obtained according to the depicted time schedule.

TABLE 1

| | Plasma 25-OH D (Change vs. baseline in nmol/L) | | |
|---|---|---|---|
| Time | | | 140 μg 25-OH D3 + |
| (hours) | 140 μg 25-OH D3 | 140 μg Vitamin D3 | 140 μg Vitamin D3 |
| 0 | 0.0 | 0.0 | 0.0 |
| 2 | 28.5 | 1.5 | 41.2 |
| 4 | 47.7 | 2.2 | 61.6 |
| 6 | 58.2 | 3.2 | 64.6 |
| 8 | 60.2 | 5.2 | 62.4 |
| 10 | 57.7 | 6.2 | 63.1 |
| 12 | 58.9 | 6.7 | 63.9 |
| 25 | 42.2 | 8.0 | 46.2 |
| 49 | 34.4 | 11.7 | 43.2 |
| 73 | 29.7 | 13.0 | 39.7 |
| 97 | 22.2 | 12.7 | 33.9 |
| 206 | 17.5 | 14.0 | 31.9 |
| 374 | 12.7 | 13.0 | 20.7 |
| 708 | 7.5 | 13.7 | 14.2 |

As shown above, there was a synergistic increase in the plasma 25-OH D response after a combined administration of 140μg 25-OH D3+140 μg Vitamin D. The effect was especially pronounced during the first 6 hours. Furthermore, a combined administration produced sustained increase in plasma 25-OH D levels by at least 30 nmol/L from 2-206 hours (i.e until 8.5 days, or over 1 week). After administration of 140 μg 25-OH D3, an increase in plasma 25-OH D levels by at least 30 nmol/L was observed between 4-49 hours only, whereas no increase of that magnitude was observed after administration of 140 μg Vitamin D alone.

Therefore, a combined administration of 140 μg 25 OH D+140 μg Vitamin D3 provides two significant advantages: It results in a rapid and synergistic plasma response of 25-OH D and it leads to an unexpectedly pronounced and long plateau of plasma 25-OH D levels. These are especially important goals of treatment of Vitamin D deficiency; fast correction of suboptimal Vitamin D status and a long and stable plasma concentration to ensure sufficient supply of all Vitamin D dependent tissues.

### Example 2

### Gene Chip Data

The objective of this study was to test the effects of Vitamin D3, 25-OH D3, and the combination of Vitamin D3 and 25-OH D3 in a skeletal muscle atrophy model using BalbC mice where tail suspension leads to skeletal muscle atrophy in the unloaded hindlimbs of the animals. Initially this model was established in rats for simulating spaceflight in humans and is commonly used in other scientific fields to study the loss of skeletal muscle mass or bone. The results are considered indicative of human conditions such as sarcopenia (degenerative loss of skeletal muscle mass and strength during the process of ageing) or immobilization of skeletal muscle (e.g. after prolonged bed rest due to fractures, surgery or trauma). Methods For our study, nine month old BalbC female mice were randomized at the beginning of the study into four groups with 10 animals per group

1. Control group: hindlimb unloaded (HU)

2. Vitamin D3 group: HU+treatment of Vitamin D3

3. 25-OH D3 group: HU+treatment of 25-OH D3

4. Vitamin D+25-OH D3 group: HU+treatment of Vitamin D3 and 25-OH D3 (combination)

The animals were placed in special cages for duration of seven days; all mice were housed separately and had free access to feed and water ad libidum. All animals were treated twice by gavage at the beginning of the experiment and 3 hours before the section:

1. the control group received vehicle (gelatine)

2. the D3 group received Vitamin D3 (50 μg/kg/bw),

3. the 25-OH D3 group received 25-OH D3 (50 μg/kg/bw)

4. the combination group received Vitamin D3+25-OH D3 (50+50 n/kg/bw)

At the end of the study the gastrocnemius muscle was taken out and directly frozen in liquid nitrogen for further analysis. To identify changes in gene expression and analyse shifts in mRNA levels in the gastrocnemius muscle we used Affymetrix Mouse 430-2 microarrays together with the version 27 (December 2008) annotation files from Affymetrix for this array type. The array contains “45,000 probe sets to analyze the expression level of more than 39,000 transcripts and variants from more than 34,000 well-characterized mouse genes and UniGene clusters” (Affymetrix, 2009).

Total RNA was isolated using the commonly used Trizol protocol. The RNA was quantified by using spectrophotometric analysis. The integrity of total RNA samples was also assessed qualitatively on an Agilent 2100 Bioanalyzer. RNA was then prepared for the one cycle cDNA synthesis. A poly-A RNA control is used for this step to provide exogenous positive controls to monitor the entire eukaryotic target labelling process. The first cDNA synthesis is done, and after the second strand cDNA synthesis the cDNA is cleaned up of double-stranded cDNA. A biotin labelled cRNA is then synthesised, cleaned up and quantified using a spectrophotometer at 260/280 nm. It is important that cRNA target is fragmented before hybridization onto a GeneChip probe arrays to obtain optimal assay sensitivity. After fragmentation the probes are hybridized on the chips (Affymetrix Mouse 430-2 chips). The chips are washed and stained in the fluidics station of Affymetrix and scanned in the gene chip scanner. The data is then transferred from the scanner for further analysis using software from Genedata (Expressionist 5.0: Refiner Array and Analyst). Data interpretation and pathway analysis was done with the online version of the GeneGo Metacore package (V5.2 build 17389).

Refiner Array evaluates microarray data for quality issues and flags problematic measurements. It provides a set of normalization algorithms and validated condensing methods to automatically pre-process and summarize raw microarray data for subsequent statistical analysis.

Analysis of microarray data revealed genes (mRNAs) which were differentially expressed between HU group and HU plus treatment groups (Vitamin D3, 25-OH D3 or combination).

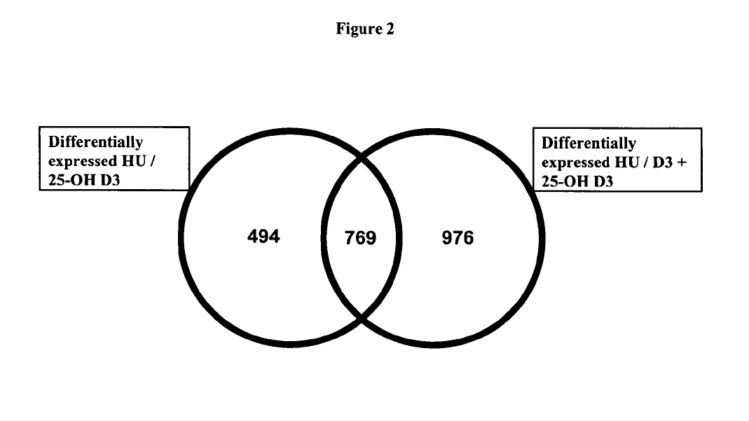

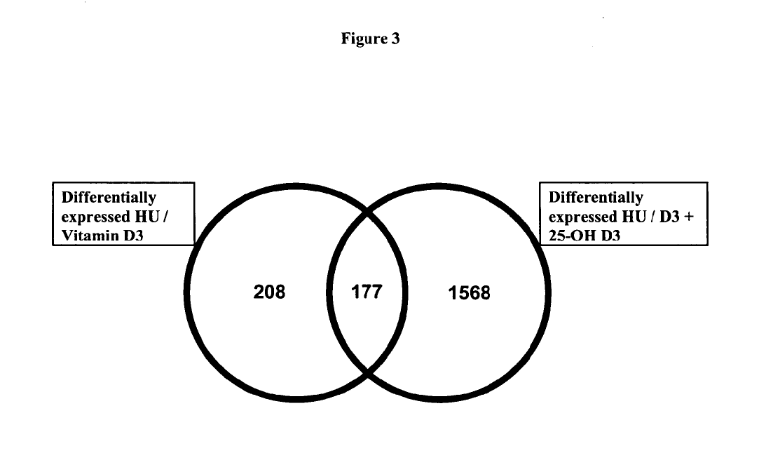

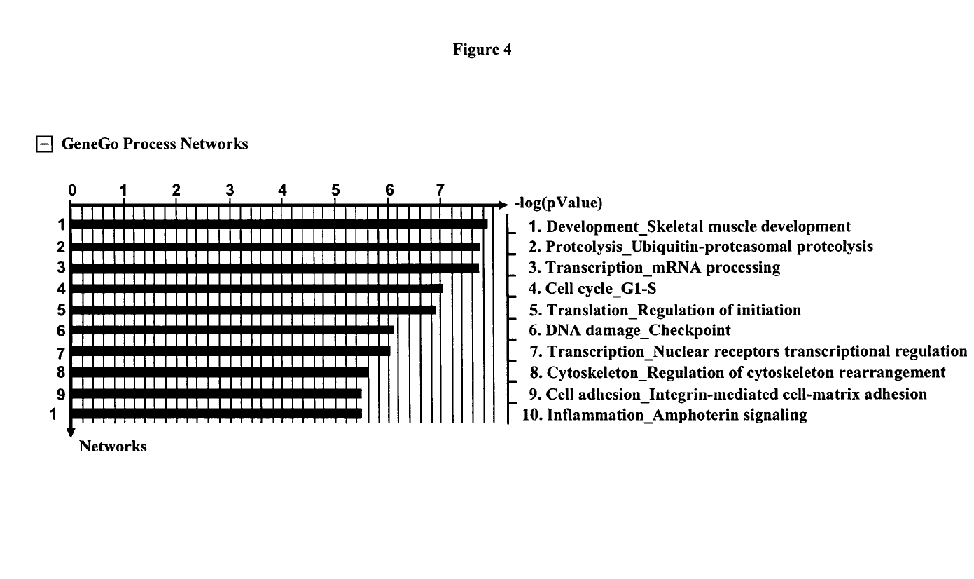

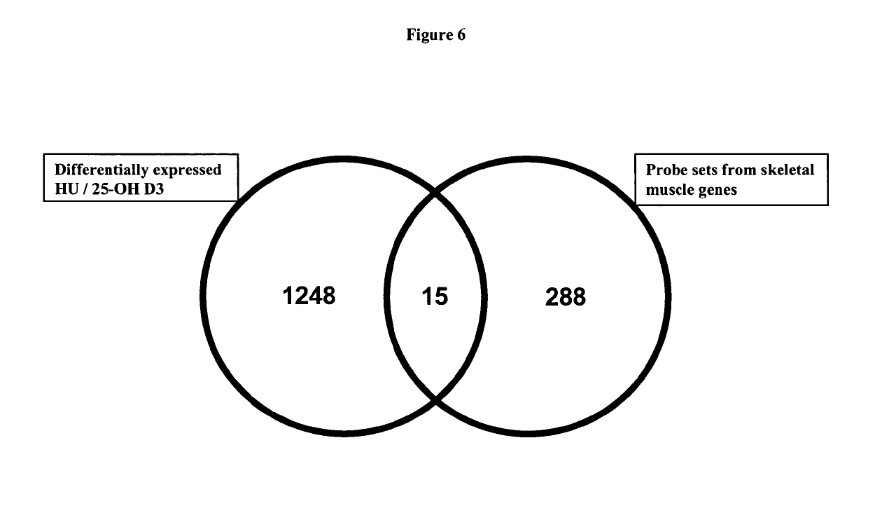

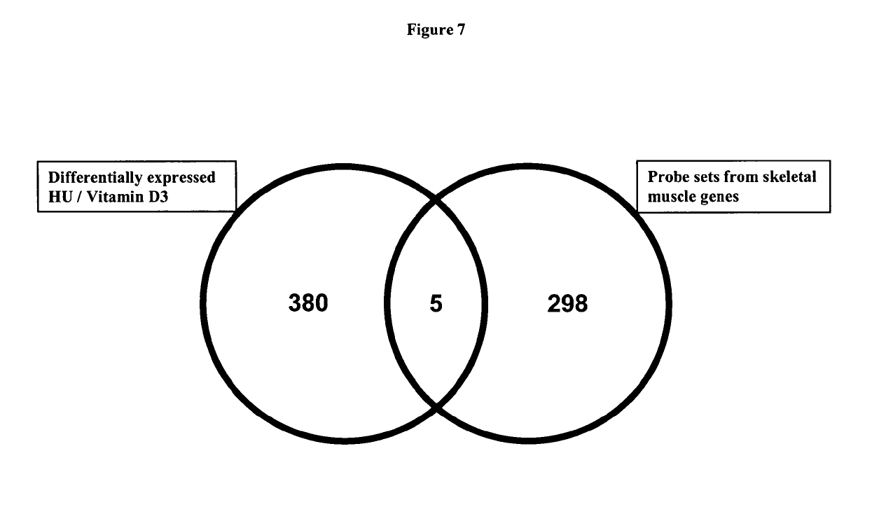

Our key findings are
1. A combination of 25-OH D3 and Vitamin D3 changes more probe sets for genes than 25-OH D3; which in turn changes more probe sets for genes than Vitamin D3 (Table 1).
   a. compared to the HU control group, the group receiving a combination treatment (D3+25-Hydroxyvitamin D3) has significantly more probe sets for genes changed (1745) than the group which received a treatment with only 25-OH D3 (1263)
   compared to the HU control group, the group receiving a treatment with 25-OH D3 has significantly more probe sets for genes changed (1263) than the group which received a treatment with Vitamin D3 (385)Error! Reference source not found.
2. A combination treatment of 25-OH D3+Vitamin D3 has more common differentially expressed probe sets for genes with 25-OH D3 treatment than with Vitamin D3 treatment.
   a. ˜61% of the probe sets for genes differentially expressed in the 25-OH D3 group are also differentially expressed in 25-OH D3+Vitamin D3 group (769 of 1263, FIG. 2)
   b. ˜46% of the probe sets for genes differentially expressed in the Vitamin D3 group are also differentially expressed in the 25-OH D3+Vitamin D3 group (177 of 385, FIG. 3)
   c. A combination treatment of 25-OH D3 and Vitamin D3 has the most significant impact on the genes of the muscle development process, as illustrated in FIGS. 4, 6, and 7.
   d. Involved genes are part of the following main categories in skeletal muscle: muscle contraction, muscle development and muscle maintenance. (Table 2).
3. For probe sets of selected genes of the skeletal muscle, a combination treatment of 25-OH D3 and Vitamin D3 shows higher expressions than treatments with only Vitamin D3 or only 25-OH D3 (
4.

5. Table 3)

TABLE 1
Differentially expressed probe sets for genes between HU and the
HU + treatment groups Vitamin D3, 25-OH D3, or combination.

| | Differentially expressed |
|---|---|
| Parameter | probe sets for genes |
| Hindlimb unloading (HU - control) group | — |
| Against HU + Vitamin D3 | 385 |
| Against HU + 25-OH D3 | 1263 |
| Against HU + Vitamin D3 + 25-OH D3 | 1745 |

TABLE 2
Differentially expressed probe sets for selected skeletal
muscle genes between HU and the treatment groups

| Parameter | Differentially expressed muscle genes compared to HU |
|---|---|
| HU + Vitamin D3 | Desmin; myoneurin; tropomyosin 1, alpha; |
| | tropomyosin 2, beta; X-linked myotubular |
| | myopathy gene 1 |
| HU + 25-OH D3 | Desmin; dystonin; myocyte enhancer factor 2A; |
| | myoneurin; myosin VIIb; myosin, light polypeptide 6, |
| | alkali, smooth muscle and non-muscle; myosin, light |
| | polypeptide kinase; myotubularin related protein 2; |
| | myotubularin related protein 4; myotubularin related |
| | protein 6; tropomyosin 2, beta; X-linked myotubular |
| | myopathy gene 1 |
| HU + Vitamin | calsequestrin 2; desmin; dystonin; dystrophin, |
| D3 + 25-OH D3 | muscular dystrophy; myocyte enhancer factor 2A; |
| | myocyte enhancer factor 2C; myocyte enhancer |
| | factor 2D; myomesin 1; myoneurin; myosin X; |
| | myosin, heavy polypeptide 6; myosin, heavy |
| | polypeptide 8; myosin, light polypeptide 6; |
| | myotubularin related protein 1; |
| | myotubularin related protein 2; myotubularin related |
| | protein 4; myotubularin related protein 6; nebulin; |
| | sarcoglycan, beta; similar to myosin, heavy |
| | polypeptide 4; titin; tropomyosin 2, beta; troponin |
| | C; troponin I; X-linked myotubular myopathy gene 1 |

TABLE 3
Expression pattern for selected genes of the skeletal muscle

| | | | | 25-OH D3 + |
|---|---|---|---|---|
| | HU | Vitamin D3 | 25-OH D3 | Vitamin D3 |
| Tropomyosin 1 | 1179 | 2199 | 2110 | 2456 |
| (Tpml1) | | | | |
| Myosin, light | 749 | 786 | 994 | 1022 |
| chain kinase | | | | |
| (Mylk) | | | | |
| Myomesin 1 | 84697 | 99099 | 105393 | 114398 |
| (Myom 1) | | | | |
| Titin (Tnt) | 11353 | 11354 | 13786 | 15647 |

The invention described and claimed herein is not to be limited in scope by the specific embodiments herein enclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In case of conflict, the present disclosure including definitions will control.

1-20. (canceled)

21. A pharmaceutical composition suitable for human use comprising Vitamin D and 25-OH D3.

22. The composition according to claim 21, further comprising
a pharmaceutically acceptable antioxidant, and
at least one pharmaceutically acceptable carrier, wherein the composition is in a tablet, capsule, or injectable form.

23. A spray dried or encapsulated beadlet composition according to claim 21.

24. Use of a pharmaceutical composition of claim 21 to make a human medicament for use in alleviating symptoms consistent with a 25-OH D deficiency.

25. A nutraceutical, food supplement or food, suitable for human use, comprising a pharmaceutical composition of claim 21.

26. A method of increasing or maintaining 25-OH D concentration in human blood, serum or plasma, or achieving a desired steady-state concentration of 25-OH D concentration in human blood, serum, or plasma, or increasing the predictability of 25-OH D concentration in human blood, serum, or plasma comprising administering Vitamin D and 25-OH D3 to a human.

27. The method according to claim 26 wherein the Vitamin D and 25-OH D3 are administered as separate dosage forms.

28. The method according to claim 26 wherein the Vitamin D and 25-OH D3 are administered in the same dosage form.

29. A method of maintaining a prolonged 25-OH D plasma level in a human of at least 60 nmol/L above baseline comprising administering a pharmaceutical composition of claim 21 to said human.

30. A method of increasing plasma levels of 25-OH D in a human by at least 30 nmol/L above baseline comprising administering a pharmaceutical composition of claim 21 for at least one week.

31. A method of rapidly increasing plasma levels of 25-OH D in a human within 2 hours comprising administrating a pharmaceutical composition of claim 21 to said human.

32. The method according to claim 26 wherein the human is elderly.

33. The method according to claim 26 wherein the a human has a malabsorption syndrome.

34. The method of claim 33 wherein said malabsorption syndrome is at least one selected from the group consisting of celiac disease, sprue, and short bowel syndrome.

35. The method according to of claim 26 wherein the human has impaired liver function.
